(19) United States
(12) Patent Application Publication
(10) Pub. No.: US 2026/0063630 A1
(43) Pub. Date: Mar. 5, 2026

(54) CAPTURE, PURIFICATION, AND RELEASE OF BIOLOGICAL SUBSTANCES USING A SURFACE COATING

(71) Applicant: Academia Sinica, Taipei (TW)

(72) Inventors: Ying-Chih Chang, Taipei (TW); Han-Chung Wu, Taipei (TW); Po-Yuan Tseng, New Taipei (TW); Jen-Chia Wu, Magong City (TW)

(21) Appl. No.: 19/060,331

(22) Filed: Feb. 21, 2025

Related U.S. Application Data
(63) Continuation of application No. 18/308,531, filed on Apr. 27, 2023, now abandoned.
Application No. 18/308,531 is a continuation of application No. 16/832,396, filed on Mar. 27, 2020, now Pat. No. 11,674,958.
Application No. 16/832,396 is a continuation of application No. 15/378,938, filed on Dec. 14, 2016, now abandoned.
Application No. 15/378,938 is a continuation of application No. 14/128,354, now Pat. No. 9,541,480.
(60) Provisional application No. 61/502,844, filed on Jun. 29, 2011.
(60) Provisional application No. 61/606,220, filed on Mar. 2, 2012.

(51) Int. Cl. G01N 33/543; C07K 16/30; C07K 17/14; G01N 1/40; G01N 33/574

(52) U.S. Cl. CPC G01N 33/54393; C07K 16/30; C07K 17/14; G01N 1/405; G01N 33/5759

(57) ABSTRACT

This invention relates to a surface coating for capture circulating rare cells, comprising a nonfouling composition to prevent the binding of non-specific cells and adsorption of serum components; a bioactive composition for binding the biological substance, such as circulating tumor cells; with or without a linker composition that binds the nonfouling and bioactive compositions. The invention also provide a surface coating for capture and purification of a biological substance, comprising a releasable composition to release the non-specific cells and other serum components; a bioactive composition for binding the biological substance, such as circulating tumor cells; with or without a linker composition that binds the releasable and bioactive compositions. The present invention also discloses a novel microfluidic chip, with specific patterned microstructures to create a flow disturbance and increase the capture rate of the biological substance.

### CROSS-REFERENCES TO RELATED APPLICATIONS

This application is a continuation of U.S. patent application Ser. No. 18/308,531, filed Apr. 27, 2023, which is a continuation of U.S. patent application Ser. No. 16/832,396, filed Mar. 27, 2020, now U.S. Pat. No. 11,674,958, which is a continuation of U.S. patent application Ser. No. 15/378,938, filed Dec. 14, 2016, which is a continuation of U.S. patent application Ser. No. 14/128,354, filed May 20, 2014, now U.S. Pat. No. 9,541,480, which is a 371 national stage entry of PCT Application No. PCT/US2012/044701, filed Jun. 28, 2012, which claims the benefit of U.S. Provisional Application No. 61/502,844, filed Jun. 29, 2011, and claims the benefit of U.S. Provisional Application No. 61/606,220, filed Mar. 2, 2012, the entirety of which are incorporated by reference.

### STATEMENT AS TO RIGHTS TO INVENTIONS MADE UNDER FEDERALLY SPONSORED RESEARCH AND DEVELOPMENT

NOT APPLICABLE

### SEQUENCE LISTING

The instant application contains a Sequence Listing which has been submitted electronically in XML format and is hereby incorporated by reference in its entirety. Said XML copy, created on Apr. 26, 2023, is named 45249.701.303_SL.xml and is 9,482 bytes in size.

### BACKGROUND OF THE INVENTION

The shedding of cells into the circulation is an intrinsic property of the malignant tumor, and this feature provides important information with regard to the diagnosis, staging, treatment response and survival of cancer patients. For example, Pantel et al found the number of circulating tumor cells (CTCs) in the blood is correlated with the aggressiveness of the cancer as well as the efficacy of the therapy. (Pantel, K. et. al., “Detection, clinical relevance and specific biological properties of disseminating tumor cells”, Nat Rev Cancer. 2008, 8(5):329-40).

However, CTCs, as few as one per 109 blood cells in patients with metastatic cancer, are rare cells. This makes the detection and isolation of CTCs technically challenging (see Kahnet al. Breast Cancer Res Treat 2004, 86:237-47). An enrichment process is therefore necessary to effectively detect and isolate CTCs.

An example of such enrichment process is the use of a highly overexpressed cell surface biomarker with high specificity and sensitivity for CTCs, such as the epithelial cell adhesion molecule (EpCAM). The Cellsearch System™ (Veridex), the only FDA-approved platform for CTC detection, utilizes anti-EpCAM antibody-coated magnetic nanoparticles to capture and enrich CTCs, followed by cytokeratin immunostaining. The AdnaTest (AdnaGen AG, Germany), another commercially available system for CTC detection, adopts similar immunomagnetic approach by using anti-EpCAM and Mucin 1 (MUC1) conjugated magnetic beads. More recently, “CTC chips” based on anti-EpCAM antibody-coated microfluidics chip were developed for CTC detection and enrichment (Nagrath et al, Nature 2007, 450:1235-9). However, the disadvantage of the above techniques is the low detection rate of pure CTCs, due to the non-specific binding of blood cells with anti-EpCAM antibody.

In order to maximize the detection and isolation of CTCs, it is necessary to reduce the nonspecific binding of other circulating blood cells. This can be achieved by surface modification with bioinert materials. For example, Kaladhar et al. observed a significant fewer circulating blood cells (e.g. platelets, leukocytes, and erythrocytes) binding onto the solid substrate modified with supported monolayer of various lipid compositions containing phosphatidyl-choline, cholesterol, and glycolipid (Kaladhar et al, Langmuir 2004, 20:11115-22 and Kaladhar et al, J Biomed Mater Res A 2006, 79A:23-35).

Despite the advance in the detection and isolation CTCs technology, there is still a need for a more specific and effective method for detecting, purificaiton and releasing CTCs and other biological substances for further cultivation and characterization.

### BRIEF SUMMARY OF THE INVENTION

In one aspect, the present invention is directed to a surface coating to capture a circulating rare cell (CRC). This surface coating increases the capture efficiency of a CRC, such as CTC, circulating stem cells (e.g. tumor stem cell and bone marrow stem cells), fetal cells, bacteria., virus, epithelial cells, endothelial cells or the like and reduces the binding of non-specific cells or protein adsorption.

The surface coating comprises 1) a nonfouling composition that reduces the binding of nonspecific blood cells and adsorption of other blood components, such as protein: and 2) a bioactive composition that captures a CRC. The surface coating further comprises a linker composition that attaches to the nonfouling composition and the bioactive composition, as illustrated in FIG. 1A.

In another aspect, the present invention is directed to a surface coating to capture and release a biological substance. This surface coating increases the capture efficiency of a biological substance, such as CTC, circulating stem cells (e.g. tumor stem cell, liver stem cells and bone marrow stem cells), fetal cells, bacteria, virus, epithelial cells, endothelial cells or the like and enhances the removal or release of the non-specific cells or protein from the surface coating.

The surface coating comprises 1) a releasable composition for releasing or removing nonspecific blood cells and other blood components, such as protein, from the surface coating; and 2) a bioactive composition that captures a biological substance. The surface coating further comprises a linker composition that attaches to the releasable composition and the bioactive composition.

The present invention is also directed to a microfluidic device, with specific microstructure designs to create a disturbed flow of blood, body fluid or biological samples to increase the capture rate of the biological substance.

The present invention is also directed to a method of manufacturing the surface coating, comprising a) forming the nonfouling or the releasable composition; and b) attaching the the linker composition with the nonfouling/releasable composition from step a) and the bioactive composition, or c) attaching the nonfouling/relesable composition from step a) with the bioactive composition.

The present invention is also directed to methods to capture and release the biological substance from the surface coating. The biological substance on the surface coating can be purified by removing the non-specific cells or protein. The captured biological substance can be released by air bubbles, ultraviolet irradiation and the like.

The present invention is also directed to uses of a biotinylated anti-EpCam antibody, EpAb4-1 antibody, to capture a CTC.

### BRIEF DESCRIPTION OF THE DRAWINGS

Embodiments of the present invention may be described with reference to the accompanying drawings.

FIG. 1A illustrates schematically an embodiment of the surface coating comprising a nonfouling composition, a linker composition and a bioactive composition.

FIG. 1B illustrates schematically the binding of a circulating tumor cell with the surface coating from FIG. 1A

FIG. 2A to FIG. 2F illustrate the chemical structures of examples of nonfouling materials.

FIG. 3 illustrates the chemical reactions of conjugation between the functional groups on the nonfouling composition and the bioactive composition.

FIG. 4A illustrates schematically the attachment of the surface coating and the solid substrate without a surface linker.

FIG. 4B and FIG. 4C illustrate schematically a linker composition with a cleavable functional group.

FIG. 4D illustrates schematically the attachment of the surface coating and the solid substrate usign a surface linker.

FIG. 5A and FIG. 5B illustrates schematically the formation of the surface coating on a solid substrate.

FIG. 6A and 6B illustrate schematically the components of a microfluidic chip.

FIG. 6C illustrates schematically the microfluidic chip assembly to capture CTCs from a biological sample.

FIG. 7A to FIG. 7H illustrate schematically the designs of the microstructures on the solid substrate.

FIG. 7I and 7J illustrate the capture efficiency of various microstructure designs in DMEM solution and blood respectively.

FIG. 8 illustrates the shear stresses of a buffer solution to release the non-specific cells and purify the captured biological substance.

FIG. 9. illustrates schematically the release of biological substance by the air bubble method.

FIG. 10A illustrates schematically the surface coating with a cleavable linker composition on a solid substrate.

FIG. 10B illustrates schematically the release of the biologic substance from the surface coating in FIG. 10A.

FIG. 11 illustrates QCM-D response of the surface coating construction.

FIG. 12 illustrates the QCM-D response of the addition of bovine serum albumin to the surface coating.

FIG. 13 are the photographs of the non-specific cells (top images) and the CTCs (bottom images) on the surface coating before and after the buffer rinse.

FIG. 14A illustrates the capture efficiency and non-specific blood cell binding of various surface coatings.

FIG. 14B are photo images which illustrate the non-specific blood cell binding of various surface coatings before and after the buffer rinse.

FIG. 15A to FIG. 15C are the photographs of the non-specific cells and the biological substance on the surface coating before and after the buffer rinse purification.

FIG. 16 illustrates the various shear stress and flushing time for the removal of HCT116 and NIH-3T3 cell populations from the surface coating.

FIG. 17 are the photographs of the CTCs released by the air bubbles.

FIG. 18 illustrates the cell cultures of the released CTCs on day 1, day 10 and day 14.

FIG. 19 illustrates schematically a CTC filtration device.

FIG. 20 illustrates the CTC binding specificity of biotinylated OC9801 antibody, biotinylated EpAb4-1 antibody, biotinylated EpCam antibody and IgG antibody.

### DETAILED DESCRIPTION OF THE INVENTION

The present invention is directed to a surface coating to effectively capture a circulating rare cell (CRC), such as CTC, circulating stem cells (e.g. tumor stem cell and bone marrow stem cells), fetal cells, bacteria, virus, epithelial cells, endothelial cells or the like.

In one embodiment, the surface coating for the capture of a CRC comprises 1) a nonfouling composition that prevents the binding of non-specific cells and adsorption of other blood components, such as protein; and 2) a bioactive composition that captures the circulating rare cells. The nonfouling composition and the bioactive composition are joined by discrete functional groups or moieties present in the nonfouling and bioactive compositions. Generally, a linkage between the two compositions is formed by an interaction comprising electrostatic interaction, hydrophilic-hydrophilic interaction, polar-polar interaction, complementary DNA binding, magnetic force, or combinations thereof.

In one group of embodiments, complementary DNA fragments are used for binding the nonfouling composition and the bioactive composition. The fragments are attached to each of the compositions and can be partially or completely complementary over their lengths. A suitable length of DNA will generally be at least. 15, 20, 25, 35, 50, 100 or more bases in length. An example of the DNA used in the present invention is an DNA tweezer. (See, B Yurke et al, A DNA-fuelled molecular machine made of DNA. Nature 2000, 406:605-608.)

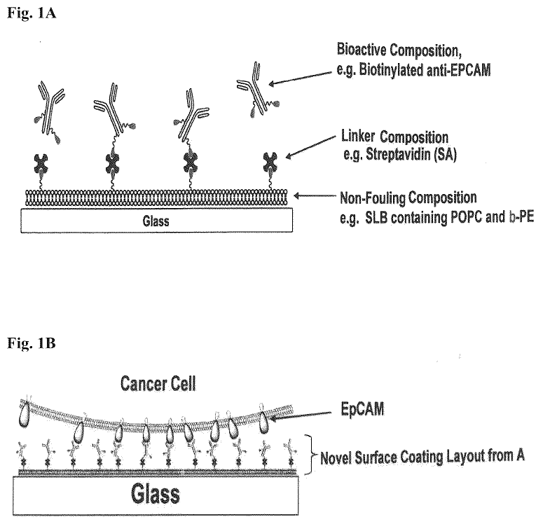

In another group of embodiments, the surface coating comprises 1) a nonfouling composition; 2) a bioactive composition; and 3) a linker composition, which joins the nonfouling composition to the bioactive composition. See FIG. 1A.

The present invention is also directed to a surface coating to effectively capture a biological substance, such as CTC, circulating stem cells (e.g. tumor stem cell, liver stem cells and bone marrow stem cells), fetal cells, bacteria, virus, epithelial cells, endothelial cells or the like, purify the biological substance on the surface of the surface coating by releasing or removing the non-specific cells and other serum components (e.g. protein) through a buffer rinse, and release the captured biological substance from the surface coating.

The surface coating for the capture and purification of a biological substance comprises 1) a releasable composition for releasing nonspecific blood cells and other blood components, such as protein, through a buffer rinse; and 2) a bioactive composition that captures a biological substance. The releasable composition and the bioactive composition are joined by discrete functional groups or moieties present in the releasable and bioactive compositions. Generally, a linkage between the two compositions is formed by an interaction comprising electrostatic interaction, hydrophilic-hydrophilic interaction, polar-polar interaction, complementary DNA binding, magnetic force, or combinations thereof.

In one embodiment, the surface coating further comprises a linker composition that attaches to the releasable composition and the bioactive composition.

As will be explained in more detail below, the surface coating can be incorporated into the following configurations: cell cultural dishes, microfluidic channels, microfluidic chips, filtration filter, capillaries, tubes, beads, nanoparticles, or the like, with an inner diameter ranging from about 50 to about 1000 um.

### Nonfouling and Releasable Composition

The “nonfouling” composition (see FIG. 1A) reduces the binding of non-specific cells and adsorption of the serum protein.

The “releasable” composition comprises a nonfouling composition which also acts as a “lubricating” surface such that only low flow shear stress is required to remove or release the non-specific cells or blood components from the surface coating, while the biological substance remains intact.

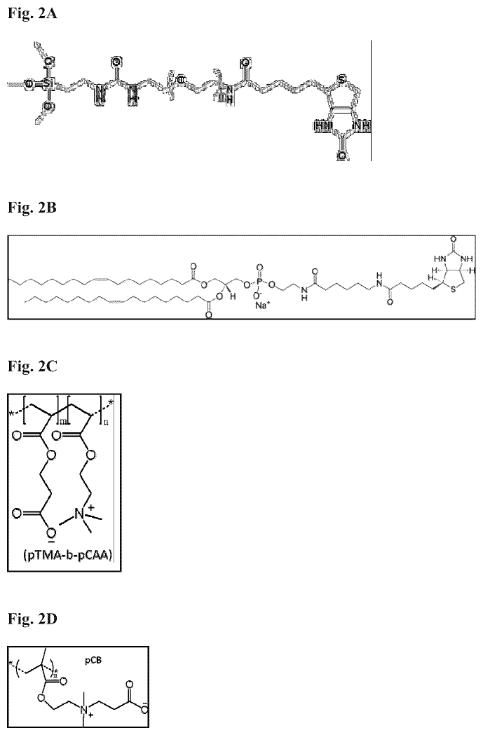

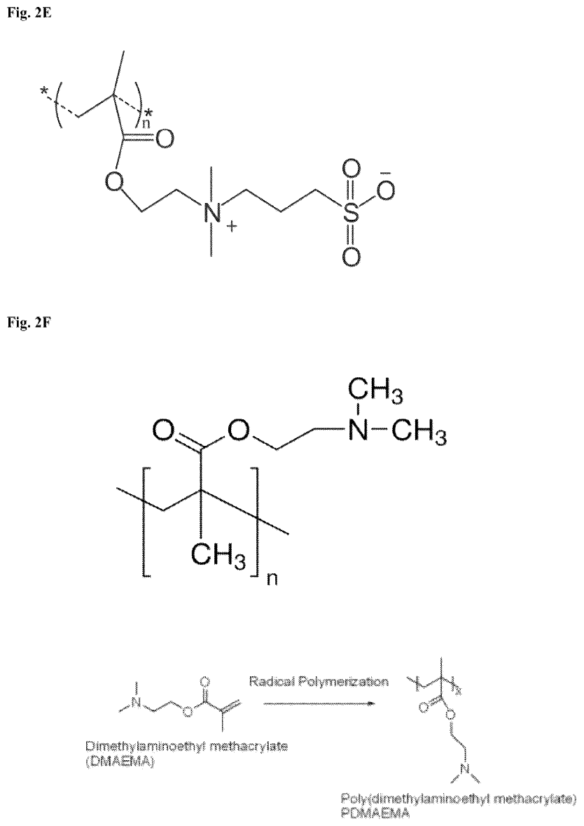

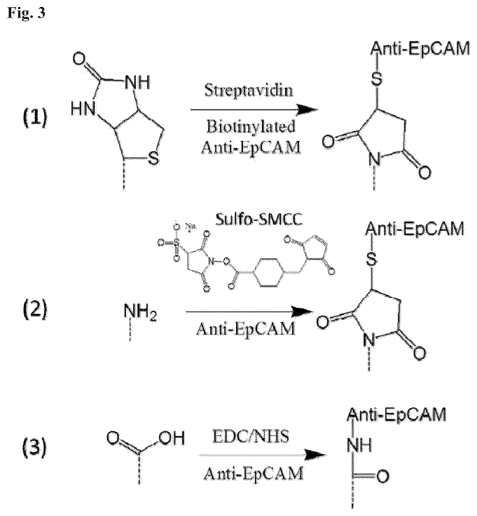

The nonfouling composition is selected from the group consisting of: a supported lipid layer such as liposomes, supported lipid bilayers (SLBs) or lipid multilayer, polypeptides, polyelectrolyte multilayers (PEMs), polyvinyl alcohol, polyethylene glycol (PEG) as illustrated in FIG. 2A, hydrogel polymers, extracellular matrix proteins, carbohydrate, polymer brushes, zwitterionic materials such as poly(carboxybetaine) (pCB)) as illustrated in FIG. 2D, poly(sulfobetaine) (pSB) as illustrated in FIG. 2E and pDMAEMA as illustrated in FIG. 2F, small organic compounds, and the combination of above materials forming a single or a multi-layer.

For those embodiments in which the nonfouling composition comprises supported lipid bilayers (SLBs), the SLBs typically comprise lipids such as, for example, 1,2-dioleoyl-sn-glycero-3-phosphoethanolamine-N-(cap biotinyl) (sodium salt) (b-PE) as illustrated in FIG. 2B and 1-Palmitoyl-2-oleoyl-sn-glycero-3-phosphocholine (POPC). The protein resistant property of a SLB can be explained by the presence of neutral and zwitterionic phosphatidylcholine headgroups in a wide pH range, as well as an aqueous thin film formed between the hydrophilic lipid head groups and the bulk solution (see, Johnson et al, Biophys J 1991, 59:289-94).

In another group of embodiments, the nonfouling composition comprises PEG, preferably PEG with a molecular weight from about 100 to about 100,000 and exhibits a nonfouling property.

In yet another group of embodiments, the nonfouling composition comprises polyelectrolyte multilayers (PEMs) or a polymer brush. Examples of suitable PEMs useful in the present invention include, but are not limited to, poly-L-lysine/poly-L-glutamic acid (PLL/PLGA), poly-L-lysine/poly-L-aspartic acid or similar counter ionic polyelectrolytes. The polymer brush comprises ([2-(acryloyloxy)ethyl] trimethyl ammonium chloride, TMA)/(2-carboxy ethyl acrylate, CAA) copolymer as illustrated in FIG. 2C. Generally, the nonfouling layer has a thickness from a few nanometers up to hundreds microns.

The nonfouling composition comprises functional groups capable of covalent, non-covalent, or a combination of covalent and non-covalent attachment, either directly to a functional group present in the bioactive composition, or directly to a functional group that is part of the linkage composition.

In some embodiments, the functional groups of the nonfouling composition (prior to covalent attachment) are selected from: hydroxy groups, amine groups, carboxylic acid or ester groups, thioester groups, aldehyde groups, epoxy or oxirane groups, hyrdrazine groups and thiol groups, which are selected to be reactive with functional groups present in either the linker or bioactive composition. In other embodiments, the functional groups of the nonfouling composition (prior to non-covalent attachment) which are first members of a binding pair, are selected from the group using specific binding recognition consisting of biotin, avidin, streptavidin, DNA, RNA, ligand, receptor, antigen, antibody and positive-negative charges, each of which is selected to bind to a second member of the binding pair which is present in either the linker or bioactive composition.

### The Linker Composition

The linker composition joins the nonfouling/releasable composition and the bioactive composition and comprises functional groups capable of covalent, non-covalent, or a combination of covalent and non-covalent attachment directly to a functional group present in the nonfouling/releasable composition and to a functional group that is part of the bioactive composition.

In some embodiments, the linker composition comprises functional groups (prior to covalent attachment) selected from: hydroxy groups, amine groups, carboxylic acid or ester groups, thioester groups, aldehyde groups, epoxy or oxirane groups, hyrdrazine groups and thiol groups, which are selected to be reactive with functional groups present in either the nonfouling or bioactive composition.

In other embodiments, the linker composition comprises functional groups (prior to non-covalent attachment) which are first members of a binding pair, selected from the group using specific binding recognition consisting of biotin, avidin, streptavidin, DNA, RNA, ligand, receptor, antigen, antibody and positive-negative charges, each of which is selected to bind to a second member of the binding pair which is present on the nonfouling/releasable composition or the bioactive composition.

The functional groups on the linker composition can also be a cleavable functional group, selected from: a photosensitive functional group cleavable by ultraviolet irradiation, an electrosensitive functional group cleavable by electro pulse mechanism, a magnetic material cleavable by the absence of the magnetic force, a polyelectrolyte material cleavable by breaking the electrostatic interaction, a DNA cleavable by hybridization, and the like.

### Bioactive Composition

The bioactive composition joins to either the linker composition or the nonfouling composition, and comprises a binding moiety selective for the detection of the biological substance or CRC.

The bioactive composition comprises functional groups capable of covalent, non-covalent, or a combination of covalent and non-covalent attachment directly to a functional group present in the nonfouling layer or to a functional group that is part of the linker composition.

In some embodiments, the functional groups of the bioactive composition (prior to covalent attachment) are selected from: hydroxy groups, amine groups, carboxylic acid or ester groups, thioester groups, aldehyde groups, epoxy or oxirane groups, hyrdrazine groups and thiol groups which are selected to be reactive with functional groups present in either the nonfouling or linker composition. In other embodiments, the functional groups of the bioactive composition (prior to non-covalent attachment) are selected from the group using specific binding recognition consisting of biotin, avidin, streptavidin, DNA, RNA, ligand, receptor, antigen-antibody and positive-negative charges, each of which is selected to bind to a second member of the binding pair which is present on the nonfouling/releasable composition or the linker composition.

The binding moiety of the bioactive composition has specific affinity with the biological substance through molecular recognition, chemical affinity, or geometrical/shape recognition. Examples of the binding moiety for the detection of the biological substance include, but.are not limited to: synthetic polymers, molecular imprinted polymers, extracellular matrix proteins, binding receptors, antibodies, DNA, RNA, antigens or any other surface markers which present high affinity to the biological substance. A preferred antibody is the anti-EpCAM membrane protein antibody (commercially available from many sources, including R&D Systems, MN, USA), which provides high specificity for CTCs because EpCAM is frequently overexpressed in the lung, colorectal, breast, prostate, head and neck, and hepatic malignancies, but is absent from haematologic cells. Another preferred antibody is Anti-HER2, which has high specificity for CTCs but absent in haematologic cells.

In one embodiment, the anti-EpCAM membrane protein antibody is EpAb4-1 antibody, compriseing a heavy chain sequence with SEQ ID No:1 and a light chain sequence with SEQ ID NO: 2 shown in Table 1.

TABLE 1
Amino Acid Sequence of V_{H} and V_{L} domains of EpAb4-1 antibody

| | FW1 | CDR1 | FW2 | CDR2 |
|---|---|---|---|---|
| SEQ ID | QIQLVQSGPELKKPGETV | GYTFTNYG | WVKQAPGKGLK | INTYTGEP |
| NO: 1 | KISCKAS | MN | WMGW | |
| (V_{H}) | | | | |
| SEQ ID | DIVMTQAAFSNPVTLGTS | RSSKSLLH | WYLQKPGQSPQ | HMSNLAS |
| NO: 2 | ASISC | SNGITYLY | LLIY | |
| (V_{L}) | | | | |
| | FW3 | CDR3 | FW4 | Family |
| SEQ ID | TYGDDFKGRFAFSLETSA | FGRSVDF | WGQGTSVTVSS | V_{H}9 |
| NO: 1 | STAYLQINNLKNEDTATY | | | |
| (V_{H}) | FCAR | | | |
| SEQ ID | GVPDRFSSSGSGTDFTLRI | AQNLENPR | FGGGTKLEIK | V_{K}24/25 |
| NO: 2 | SRVEAEDVGIYYC | T | | |
| (V_{L}) | | | | |

Complementary-determining regions 1-3 (CDR1-3), framework regions 1-4 (FW1-4) for both the V_{H} and V_{L}, domains are shown. The V domain families were aligned by VBASE2 database (www.vbase2.org).

The bioactive composition can have a variety of thicknesses, selected so that it does not affect the function or the performance of the surface coating.

In one embodiment, the conjugation linkers or catalysts for the nonfouling composition and the bioactive compositions are biotin/avidin or their derivatives. In another embodiment, the conjugation linkers or catalysts for the nonfouling composition and the bioactive composition are EDC/NHS. In yet another preferred embodiment, the conjugation linkers or catalysts for the nonfouling composition and the bioactive compositions are sulfo-SMCC. FIG. 3 schematically illustrates the chemical reactions of these embodiments.

### Solid Substrate

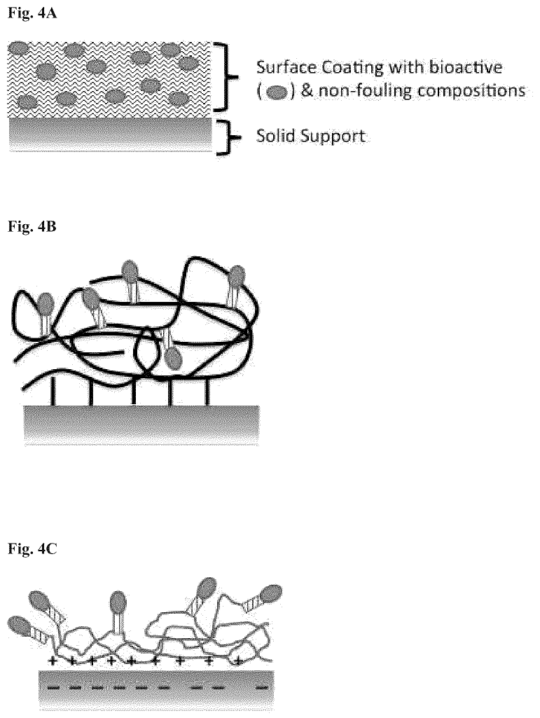

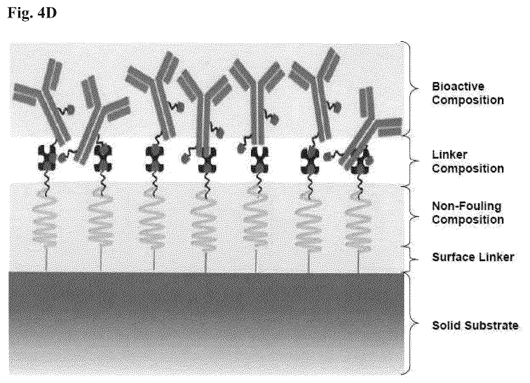

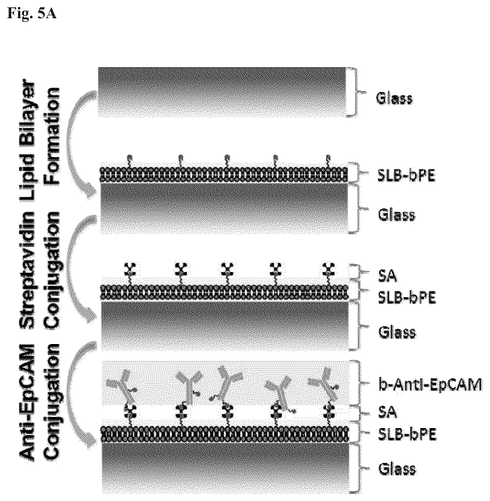

In some embodiments, the surface coating is attached to the solid substrate without a surface linker, as illustrated in FIG. 4A. The nonfouling/releasable composition is attached to the solid substrate via one of the following interactions: covalent bonding (for PEG nonfouling composition), hydrogen bonding, electrostatic interaction, hydrophilic-hydrophilic interaction (for SLB nonfouling/releasable composition), polar-polar interaction, complimentary DNA binding, magnetic force, or the like.

In other embodiments, the surface coating is attached to the solid substrate with a surface linker, as illustrated in FIG. 4D. Examples of the solid substrate used in the present invention include, but are not limited to: metals, plastics, glass, silicon wafers, hydroxylated poly(methyl methacrylate) (PMMA), and a combination thereof. The shape of the solid substrate include, but are not limited to: planar, circular and irregular shapes with micro, or nano-structures such as nanoparticles, nanowires, and a combination thereof

The surface linker composition comprises functional groups capable of covalent, non-covalent, or a combination of covalent and non-covalent attachment directly to a functional group present in the nonfouling/releasable composition and to a functional group that is part of the solid substrate. Examples of the surface linker for binding the surface coating to a glass substrate include, but are not limited to, silane, aminopropyltriethoxy silane, aminopropyltrimethoxy silane, silane-PEG-NH₂, silane-PEG-N₃ (PEG molecular weight is about 1,000 to about 30,000 daltons) and silane-PEG biotin.

In one group of embodiments, the surface linker comprises a cleavable functional group selected from: a photosensitive functional group cleavable by ultraviolet irradiation, an electrosensitive functional group cleavable by electro-pulse mechanism, an iron or magnetic material in which the absence of the magnetic force will release the nonfouling composition, a polyelectrolyte material cleavable by breaking the electrostatic interaction, an DMA cleavable by hybridization, and the like.

In one embodiment, the nonfouling composition comprises silane-functionalized PEG and the solid substrate is preferably selected from the group consisting of silicon, glass, hydroxylated poly(methyl methacrylate) (PMMA), aluminum oxide, TiO₂ and the like. In another embodiment, the nonfouling composition comprises thiol-functionalized compounds and the solid substrate is preferably selected from the group consisting of Au, Ag, Pt, and the like.

### The Method of Manufacturing the Surface Coating

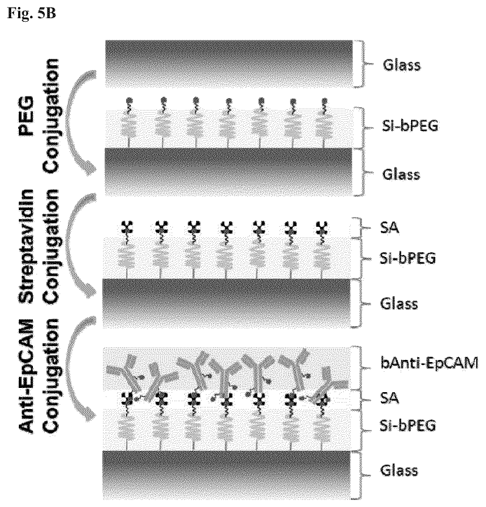

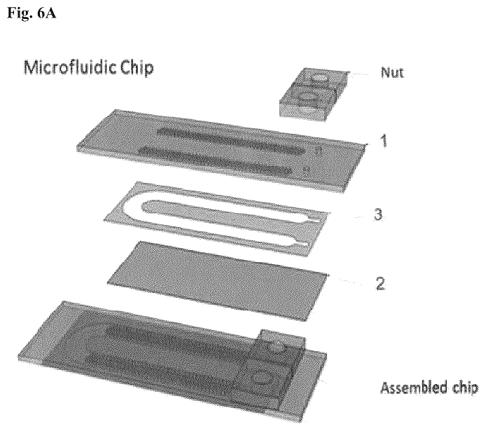

FIGS. 5A and 5B show the steps of forming the surface coating:
1. Formation of the nonfouling/releasable composition (e.g. SLB or PEG) with appropriate functional group (biotin);
2. Attaching the functional group (streptavidin) on the linker composition to the functional group (biotin) on the nonfouling/releasable composition;
3. Formation of the bioactive composition and attaching the functional group (biotin) on the bioactive composition to the functional group (streptavidin) on the linker composition.

The surface coating without a linker composition can be formed by:
1. Formation of the nonfouling/releasable composition with appropriate functional group (e.g. carboxyl group of N-glutaryl phosphatidylethanolamine or NGPE);
2. Formation and attaching the functional group (primary amine) on the bioactive composition to the functional group (carboxyl group of NGPE) on the nonfouling/releasable composition in step 1.

The steps in paragraphs [0077] and [0078] can be reversed.

### Microfluidic Chip

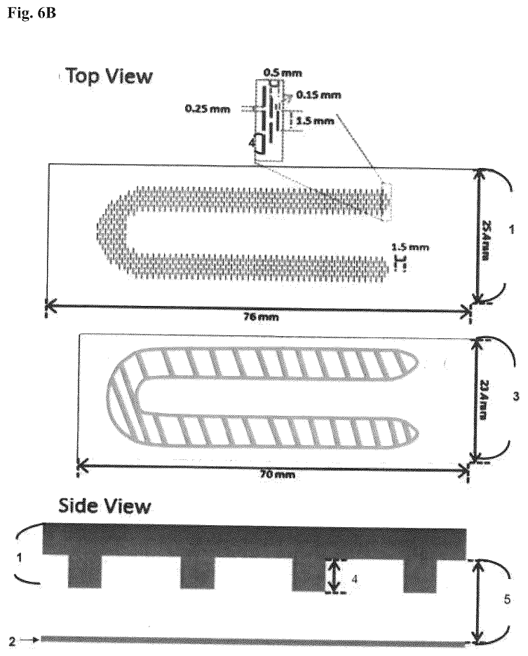

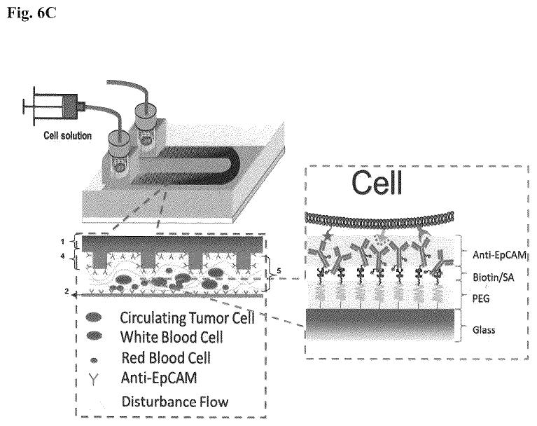

As illustrated in FIG. 6A, the microfluidic chip comprises a first solid substrate 1 (e.g. PMMA) and a second solid substrate 2 (e.g. glass), wherein the first and second solid substrates are adhered together using an adhesive means 3 or other means.

Referring to FIG. 6B, the surface of one or both solid substrates can be engraved with microstructures 4. In one group of embodiments, the microstructures 4 are arranged in a linear fashion. In another group of embodiments, the microstructures 4 are arranged in herringbone fashion. The shaded region on the adhesive 3 in FIG. 6B is carved out to accommodate the microstructures 4 on the surface of the solid substrate 1. A sealed channel 5 is created by adhering the first solid substrate 1 and the second solid substrate 2 together with an adhesive 3. The height of the channel 5 is determined by the thickness of the adhesive 3.

Once the microfluidic chip is formed, the surface coating can be attached to one or both solid substrates. In one group of embodiments, the surface coating is attached to the solid substrate with a surface linker. In another group of embodiments, the surface coating is attached to the solid substrate via one of the following interactions: covalent bonding (for PEG nonfouling composition), hydrogen bonding, electrostatic interaction, hydrophilic-hydrophilic interaction (for SLB nonfouling/releasable composition), polar-polar interaction, complimentary DNA binding, magnetic force, or the like.

Referring to FIG. 6C, the microstructures 4 on the solid substrate 1 are perpendicular to the flow direction and create a chaotic or disturbed flow of the blood, body fluid or biologic sample as it passes through the sealed channel 5 of the microfluidic chip. The disturbed flow enhances the biological substance-surface coating contact.

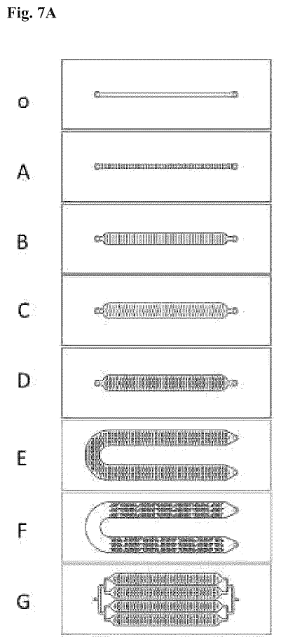

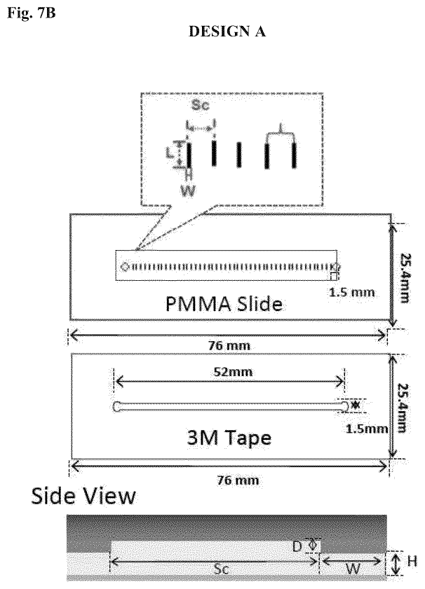

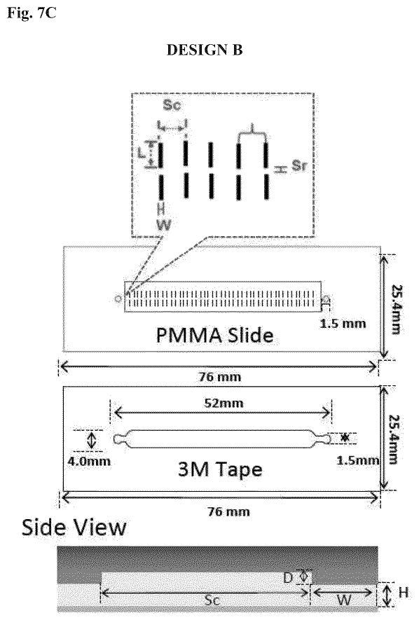

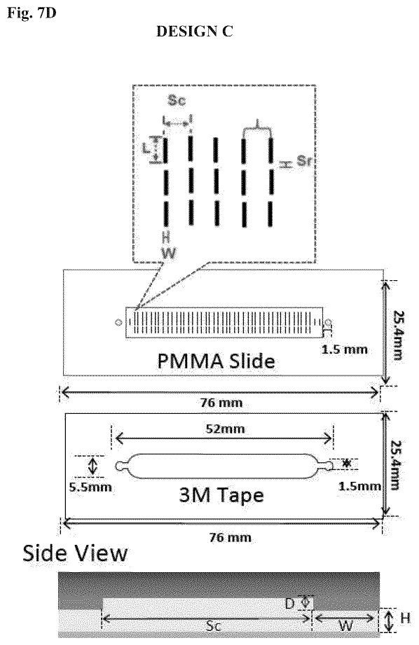

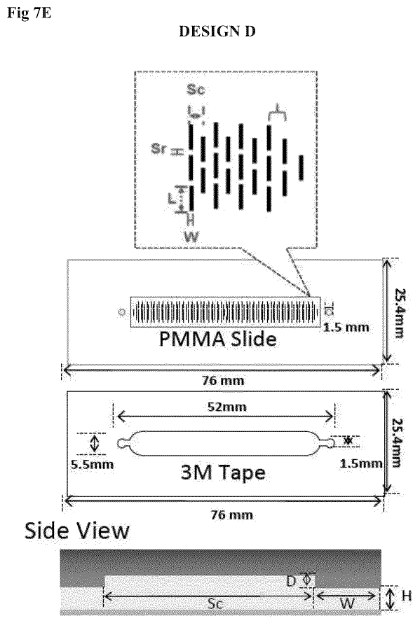

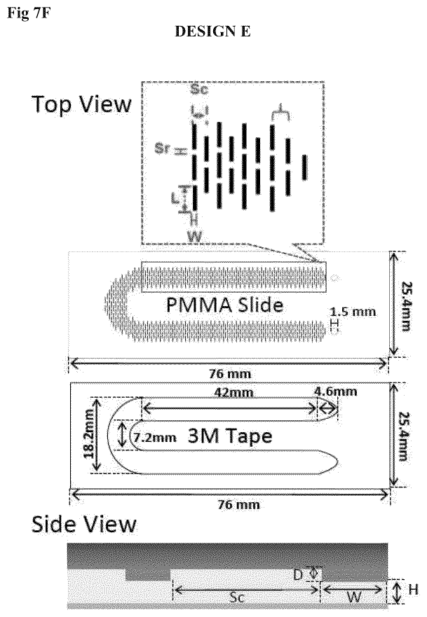

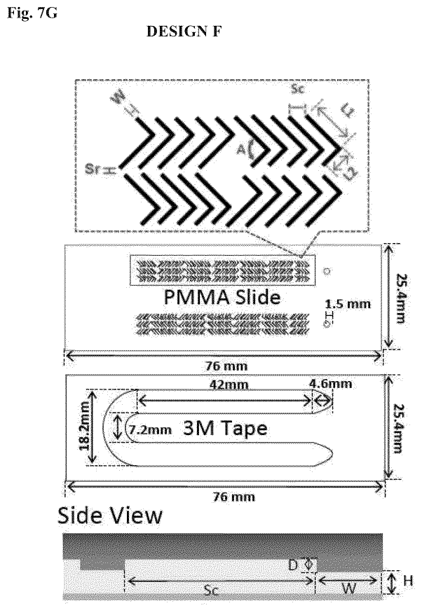

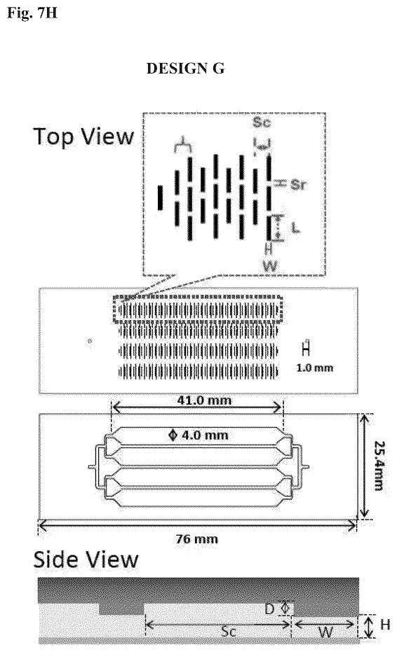

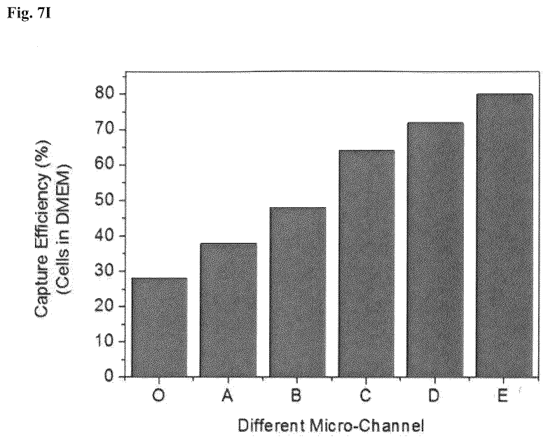

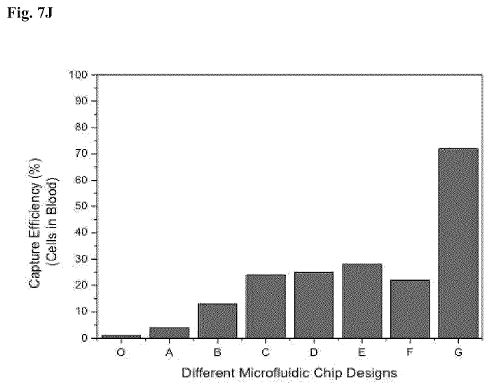

Two factors govern the capture effeiciency of the microfluidic chip:
(1) The linear speed of the blood, body fluid or biological sample, which determines the contact time of the biological substance and the surface coating. In a preferred embodiment, the linear speed is about 0.1 mm/s to 1 mm/s. In a more preferred embodiment, the linear speed is about 0.42 mm/s or 0.5 ml/h for Design E in FIG. 7F.
(2) The flow disturbance of the blood, body fluid or biological sample, created by the microstructures 4 on the solid substrate(s). The flow disturbance increases contact between the biological substance and the surface coating.

FIG. 7A shows various designs of the microstructures 4 on the solid substrate. The microstructures in Design F are arranged in a herringbone pattern whereas the microstructures in Designs A-E and H are arranged in a linear pattern. The dimensions of the microstructures 4 are as follows: the length is about 50 mm for O-D and G and about 120 mm for E-F, the height is about 30 μm the width is about 1.5 mm for O and A, about 3.0 mm for B, and about 5.5 mm for C-G. The height of the sealed channel 5 varies with the thickness of the adhesive 3, preferably about 30-90 μm, more preferably about 60 μm.

FIG. 7B-7H show the details of Designs A-G in FIG. 7A. Design G in FIG. 7H is the preferred pattern, with the following dimensions: the width of Microstructure (W) is about 150 μm, the length of microstructure (L) is about 1000 μm, the distance between two rows of microstructures (Sr) is about 250 μm, the distance between two adjacent microstructures (Sc) is about 350 μm, the height of the microstructure (D) is about 30 μm and the height of the sealed channel 5 (H) is about 60 μm.

The biological substance capture efficiency of the various designs are shown in FIG. 7I and FIG. 7J. Capture rate is defined as (captured biological substance/original biological substance in the testing sample)×100%. Channel O has no microstructure and has the lowest biological substance capture rate, at 27% and 1% for DMEM sample and blood sample, respectively. Design E has a 80% capture rate for HCT116 cancer cells spiked in DMEM, and a 30% capture rate for HCT116 cancer cells spiked in blood sample. Design F has the best capture rate, on average over 70% of HCT116 cancer cells spiked in blood sample were captured (see FIG. 7J).

### Flow Purification

The biological substance on the surface coating can be further purified by removing the non-specific cells and other blood components on the surface of the nonfouling/releasable composition. The nonfouling/releasable composition has low affinity for non-specific cells and other blood components. Therefore, rinsing the surface coating with a low flow buffer solution of about 0.8 dyne/cm² to about 50 dyne/cm² is sufficient to remove non-specific cells and other blood components on the nonfouling/releasable composition while the biological substance remains on the surface coating.

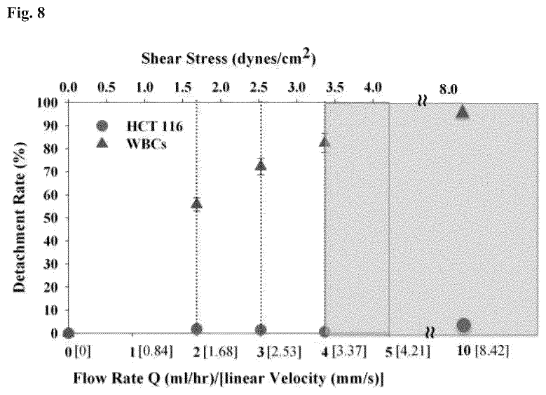

In a preferred embodiment, the shear force of the buffer rinse is about 2.5 to about 10 dyne/cm² FIG. 8 shows that when the shear stress of the buffer flow is about 3.3 dyne/cm², 80% of the non-specific cells (i.e. white blood cells) were removed while none of the biological substance (i.e. HCT 116 cancer cells) were removed from the surface coating. When the shear stress of the buffer flow was increased to 8 dyne/cm², almost all of the non-specific cells were removed while none of the biological substance was removed from the surface coating.

### Release of the Biological Substance

After removing the majority of the non-specific cells and blood components by flow purification, the biological substance can be released from the surface coating.

If the nonfouling/releasable composition comprises a lipid or a mixture of lipid, the captured biological substance can be released by introducing an air bubble solution or oil phase. As shown in FIG. 9, the surface coating comprises a nonfouling composition A (lipid bilayer) and a bioactive composition B (antibody) and is bound to a solid substrate S. The biological substance, CTC, is bound to the bioactive composition B, whereas other cells were repelled by the nonfouling composition A. As the air bubble approaches the lipid bilayer, the hydrophobic tails of the lipid bilayer are turned upside down due to its high affinity with the air inside the air bubble, which is also hydrophobic. This breaks up the hydrophilic-hydrophilic interaction at the surface of the lipid bilayer and allows the air bubble to “lift off” the top layer of the lipid bilayer, together with the CTC bound on the bioactive composition.

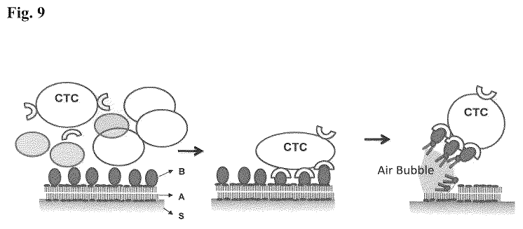

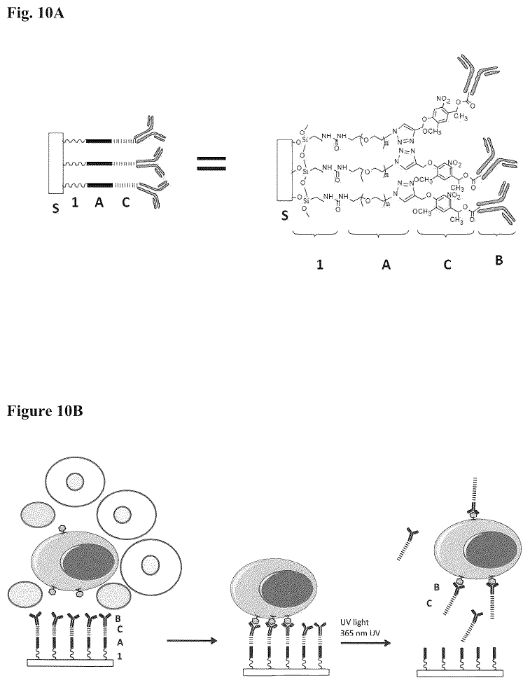

If the nonfouling composition comprises a composition other than a lipid or a mixture of lipid, the captured biological substance can be released by breaking the cleavable functional group on the linker composition or on the surface linker. This release mechanism is illustrated in FIGS. 10A and 10B. FIG. 10A shows a surface coating on a solid substrate, wherein the surface coating comprises a bioactive composition B, a linker composition with a cleavable functional group C, and a nonfouling composition A. The surface coating is attached to a solid substrate S (e.g. glass) by a surface linker 1. FIG. 10B shows the release of the biologic substance (e.g. CTC) from the surface coating in FIG. 10A. The biologic substance is bound to the bioactive composition B, whereas other cells were repelled by the nonfouling composition A. The surface coating is irradiated with 365 nm ultraviolet light, which breaks the cleavable functional group on the linker composition C and the biologic substance is released for subsequent analysis but maintaining the viability.

The biological substance can also be released by other mechanisms. In one group of embodiments, the linker composition or the surface linker comprises an electrosensitive cleavable functional group, and the biological substance is released by electro pulse mechanism. In another group of embodiments, the linker composition or the surface linker comprises a magnetic material as the cleavable functional group, and the absence of the magnetic field or force releases the biological substance. In yet another group of embodiments, the linker composition or the surface linker comprises a PEM as the cleavable functional group, and the biological substance is released by changing the electrostatic interaction between the layers. In yet another group of embodiments, the linker composition or the surface linker comprises an DNA piece as the cleavable functional group, and the biological substance is released by DNA hybridization.

### EXAMPLES

The following examples further illustrate the present invention. These examples are intended merely to be illustrative of the present invention and are not to be construed as being limiting.

### Example 1: Preparation of the Two-Layer Surface Coating

### Preparation of the Nonfouling Composition:

Supported lipid bilayer (SLB) was prepared by the following steps:
(1) POPC and b-PE (commercially available from Avanti Polar Lipids, USA) were dissolved in chloroform and the final lipid concentration was 5 mg/mL. The POPC/b-PE solution was vortex dried under a slow stream of nitrogen to form a thin, uniform POPC/b-PE film. The POPC/b-PE film was further dried in a vacuum chamber overnight to remove residual chloroform.
(2) The POPC/biotin-PE film in step (1) was dispersed in and mixed with a phosphate buffer containing 10 mM of phosphate buffered saline, 150 mM of sodium chloride aqueous solution, and 0.02% (w/v) of sodium azide (NaN₃, commercially available from Sigma-Aldrich, USA), with the pH adjusted to 7.2. The mixed solution was filtered through the 100-nm, followed by the 50-nm Nuclepore® track-etched polycarbonate membranes (Whatman Schleicher & Schuell, Germany) at least 10 times under 150 psi at room temp.
(3) The filtered solution in step (2) was passed through the LIPEX™ Extruder (Northern Lipids, Inc. Canada) to generate a homogenous population of unilamillar vesicles. The size of the POPC/biotin-PE vesicles was about 65±3 nm, determined by the dynamic laser light scattering detector (Zetasizer Nano ZS, Malvern Instruments, Germany).

### Preparation of the Bioactive Composition

Biotinylated EpCAM Antibody was prepared by the following steps:
(1) The anti-EpCAM monoclonal antibody (OC98-1 or EpAb4-1) was generated by method described by Chen et al (Clin Vaccine Immunol 2007; 14:404-11).
(2) The antibody in step (1) was dissolved in a buffer solution containing 10 mM of PBS and 150 mM of NaCl, with a pH about 7.2. The concentration of the antibody buffer solution was about 0.65 mg/mL, determined by Nanodrop 1000 spectrophotometer (Thermo Scientific, USA).
(3) The antibody solution in step (2) was mixed with 10 mM of Sulfo NHS-LC-Biotin (with a molar ratio of 1 to 10) and dissolved in Milli-Q water (Milli-Q RO system, USA) at room temperature for 30 min. Excess biotin was removed by dialysis in phosphate buffered saline at 4° C. for 24 h, with a buffer change every 12 h.
(4) The ratio of biotin and antibody in the biotinylated anti-EpCAM antibody (bOC98-1 or bEpAb4-1) was 1.5 to 1, determined by the HABA assay using a biotin quantitation kit (Pierce, USA).

Alternatively, commercially available biotinylated goat anti-human anti-EpCAM antibody from R and D Systems (Minneapolis, MN) could be used.

### Preparation of Solid Substrates of the Present Invention

Glass substrate (such as microscope coverslips from Deckglaser, Germany) were cleaned with 10% DECON 90 (Decon Laboratories Limited, England), rinsed with Milli-Q water, dried under nitrogen gas, and exposed to oxygen plasma in a plasma cleaner (Harrick Plasma, Ithaca, NY, U.S.A.) at 100 mtorr for 10 min. Prior to each use, the glass substrate was rinsed with ethanol and dried under nitrogen gas.

Silicon oxide based solid substrates (e.g. silicon wafer or glass coverslips) were cleaned with piranha solution (70% sulfuric acid and 30% hydrogen peroxide (v/v)) at 120° C. for 40 min, subsequently washed with distilled water and rinsed with acetone. The solid substrates were dried under a stream of nitrogen and treated with a plasma cleaner.

For the vapor phase silanization reaction, clean silicon oxide substrates and a Petri-dish containing 150 uL of 3-(aminopropyl)-triethoxysilane (Sigma, USA) were placed in a desiccator (Wheaton dry-seal desiccator, 100 nm) under reduced pressure at ˜0.3 Torr for 16h. The substrates were cleaned by acetone and dried under nitrogen stream.

### Construction of the SLB Surface Coating on a Solid Substrate

0.25 mg/ml of POPC/b-PE vesicle solution from paragraph was added to the cleaned solid substrate to form a SLB coated solid substrate. This was followed by an extensive rinse with a phosphate buffer containing 10 mM PBS and 150 mM NaCl (pH=7.2) to remove excess POPC/b-PE vesicles. Biotin was the functional group in the SLB which binds with the functional group (streptavidin) in the linker composition

0.1 mg/mL of streptavidin (SA) solution (commercially available from Pierce Biotechnology, Rockford, IL, USA) was added to the SLB coated solid substrate and incubated for 1 hour, followed with a PBS buffer rinse to remove excess SA.

About 0.05 mg/mL of b-Anti-EpCAM solution was added to the SA-SLB coated solid substrate to form the surface coating of the present, invention.

### Construction of the PEG Surface Coating on a Solid Substrate

The biotinylated PEG silane solution (Si-bPEGs) was added to the clean glass substrate and incubated for 1 hour to form a Si-bPEG nonfouling composition on the glass substrate, followed by an ethanol rinse to remove excess Si-bPEGs. Silane was the surface linker and the biotin was the functional group that bind with the functional group (SA) in the linker composition.

0.1 mg/mL of SA solution was added to the Si-bPEGs coated solid substrate and incubated for 1 hour, followed by a PBS buffer rinse to remove excess SA.

0.05 mg/mL of b-Anti-EpCAM solution was added and bound with SA-Si-bPEGs surface coating, followed by PBS buffer rinse to remove excess b-Anti-EpCAM.

### Construction of the PEM Surface Coating on a Solid Substrate

Physical deposition of PEM films was performed by batch and static conditions as follows: initially, all polypeptides were dissolved in 10 mM Tris-HCl buffer with 0.15 M. NaCl, pH 7.4. Solid substrates were then immersed in PLL (MW 15000-30000; Sigma, St Louis, MO) solution (1 mg/mL) for 10 min at room temperature, followed by rinsing with 1 mL of Tris-HCl buffer for 1 min. To couple PLGA, the PLL-coated slide was subsequently immersed in the PLGA solution (MW 3000-15000, Sigma, St Louis, MO, 1 mg/mL) for 10 min, followed by rinsing with 1 mL of Tris-HCl buffer for 1 min. Lastly, substrates were cleaned with fresh PBS to remove uncoupled polypeptides. The resulting c-(PLL/PLGA)i, where i was denoted as the number of polyelectrolyte pairs generated by repeating the above steps: i) 0.5 was referred to c-PLL only, i) 1 was referred to c-(PLL/PLGA)1, and the like.

### QCM-D Characterization of the SLB Surface Coating

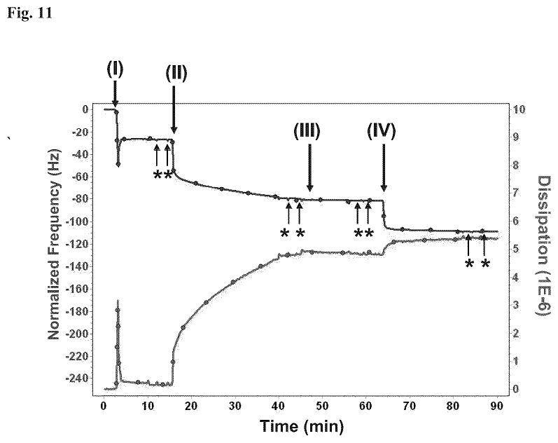

The construction of the surface coating was monitored by quartz crystal microbalance with dissipation (QCM-D). The QCM-D response in FIG. 11 shows the construction of the surface coating on a SiO₂-pretreated quartz crystal. First, 0.25 mg/mL of POPC/b-PE vesicle mixture (in phosphate buffer) was dispensed into the QCM chamber at point (I). The normalized frequency change F and dissipation shift D were 26.0±0.7 Hz and 0.19±0.03×10−6 respectively, which are the characteristics of a highly uniformed lipid bilayer. After two buffer washes (denoted as *), 0.1 mg/mL of SA solution was dispensed at point II. ⋅SA binding was saturated at F=52.8+5.4 Hz and D=3.84+0.54×10−6. At point (III), 0.025 mg/mL of OC98-1 antibody solution was dispensed into the QCM chamber and there was no frequency or dissipation change. This shows there was no interaction between the OC98-1 antibody and the SA-lipid bilayer surface. In contrast, adding biotinylated antibody solution (bOC98-1 orbEpAb4-1) at point (IV) resulted in frequency and dissipation change, with equilibrated shifts of F=39.4±6.8 Hz and D=1.63±0.28×10−6. This demonstrates the binding of biotinylated antibody to SA-lipid bilayer surface.

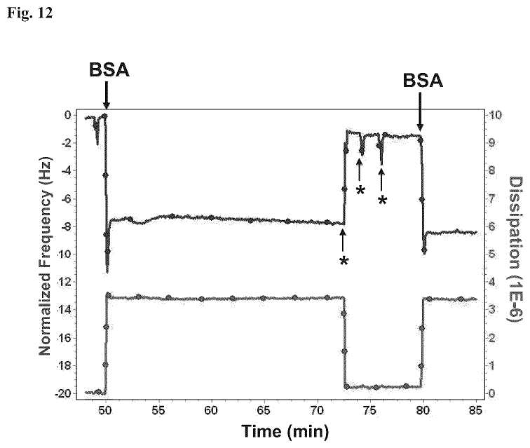

The characteristics of the SLB nonfouling composition on the surface coating were examined using QCM-D (FIG. 12). Bovine serum albumin (BSA, commercially available from Sigma-Aldrich, USA) was added to the surface coating and there was a sudden change in frequency and dissipation, with equilibrated shifts of F=6.9 Hz and D=3.35×10−6. This indicates an immediate BSA adsorption. Three buffer rinses (*) caused an increase in frequency and a decrease in dissipation, with saturated shifts of F=6.1 Hz and D=3.16×10−6. This indicates the adsorbed BSA can be easily removed from the surface coating and thus, a very weak interaction between BSA and SLB.

### Example 2: Preparation of the Microfluidic Chip

The microfluidic chip can be prepared by the following steps:
1. A commercial CO₂ laser scriber (Helix 24, Epilog, USA) was used to engrave the microtrenches to form microstructures on the PMMA substrate.
2. The PMMA substrate, glass substrate and nuts were cleaned with MeOH, detergent and water, followed by 10 min sonication. The nuts and the solid substrates were dried by nitrogen gas and baked for 10 min at 60° C.
3. The PMMA substrate was bonded with nuts by chloroform treatment.
4. PMMA substrate and the glass slide were joined together using an adhesive (e.g. 3M doubled sided tape from 3M, USA).

### Example 3: CTCs Binding to the Anti-EpCAM functionalized SLB Surface coating

Eight blood samples were used to determine the CTC capture rate of the Anti-EpCAM functionalized SLB surface coating in a microfluidic chip in Example 2. Each blood sample contained 2 ml of blood from a stage IV colon cancer patient and the sample was introduced to the sealed channel of the microfluidic chip at 0.5 ml/hr, controlled by a syringe pump. Subsequently, the sealed channel in the microfluidic chip was rinsed with 0.5 ml of PBS buffer at the flow rate of 1 ml/h, followed by in situ immunostaining.

The number of CTCs captured per ml of blood for these 8 samples were 26, 34, 36, 39, 47, 67 79, and 99. 25% of the blood samples had 79 or higher CTC count per ml of testing sample and the median CTC count was 43 per ml of testing sample. There was minimal binding of the non-specific cells and proteins after the buffer rinse.

As a comparison, the CTC count for the FDA approved Veridex CellSearch is as follows: 25% of the samples had 3 or more CTCs per 7.5 ml of testing sample and the median CTC counts was 0.

The anti-EpCAM functionalized SLB surface was incubated with 150 uL of HCT116 cancer cell spiked human blood (with HCT116 cancer cell density of approximately 10 to 100 per 100 μL of blood), followed by a buffer rinse to remove non-specific cells. FIG. 13 shows the surface coating before and after the buffer rinse. Prior to the buffer rinse, the surface coating was covered with non-specific cells (upper left) and four HCT116 cancer cells (lower left). After the buffer rinse, almost all of the non-specific cells were removed (upper right) but the four HCT116 cancer cell (lower right) remained on the surface coating.

The results show the surface coating of the present invention is effective in capturing CTCs and releasing the non-specific cells.

### Example 4: Comparison of Capture Efficiency and Nonfouling Property of Various Surface Conditions

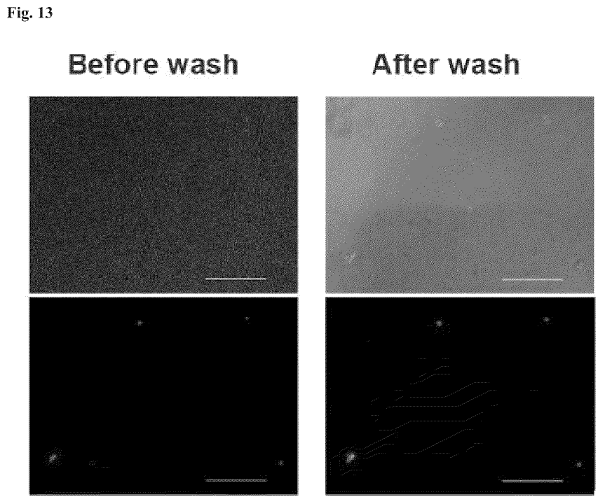

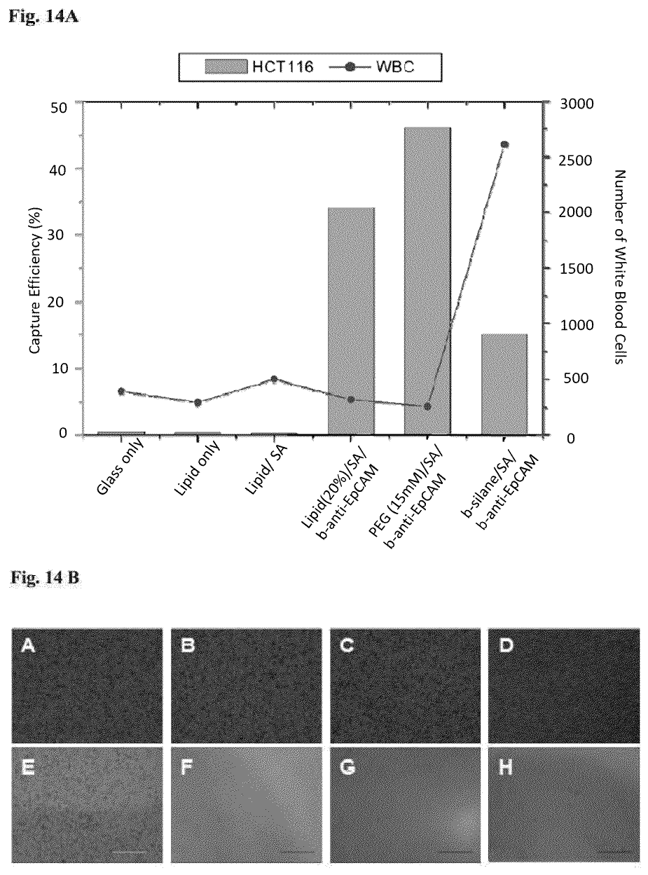

The capture rate of HCT116 cancer cells (biological substance) and the nonfouling property of six different surface conditions are illustrated in FIG. 14A.

The results show that the surface coatings of the present invention (lipid/SA/b-anti-EpCAM and PEG(15 mM)/SA/b-anti-EpCAM) are more effective in capturing the biological substance. There is less binding of the non-specific cells (white blood cells or WBC) on the surface coatings of the present invention compare to a surface coating without a nonfouling composition (glass only).

FIG. 14B shows the non-specific blood cell binding of the following surfaces: (A) Glass only; (B) biotinylated SLB (b-SLB), (C) Streptavidin conjugated-bSLB, and (D) OC98-1-conjugated bSLB. These surfaces were incubated with diluted human blood from healthy donor (1 uL of blood in 100 uL PBS buffer) for 4 hours, followed by a PBS buffer rinse. Images (E) to (H) are the after rinse images which correspond to the surface coatings in (A) to (D). The results show that after a buffer rinse, there is less non-specific blood cell on the surface coatings with a releasable composition (i.e. SLB) compare to the surface coating without a releasable composition (i.e. glass only).

### Example 5: Purification by Flow

The differentiated flow shear could selectively “flush” out the non-specific cells based on the affinity of these cells to the nonfouling composition, while the biological substance remains on the surface coating.

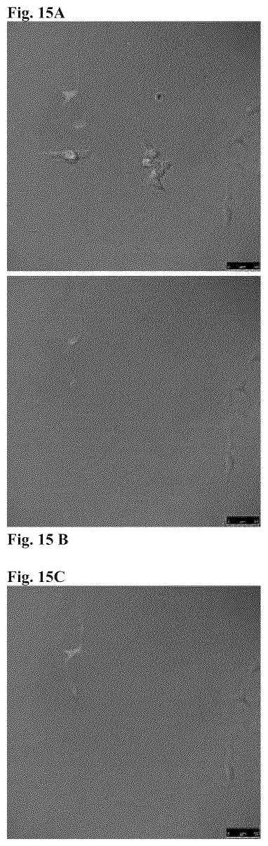

In this study, the surface coating comprised a SLB, a linker composition and fibronectin as the bioactive composition. FIG. 15A shows fibroblast 3T3 (green) and colon cancer cell line HCT116 (red) were incubated on the surface coating for 4 h. The surface coating was rinsed with a buffer solution, which has a shear stress of 3 dyne/cm².

The HCT 116 cells (red) were flushed away from the surface coating within 5 min of the buffer rinse, as shown in FIG. 15B. The fibroblast 3T3 cells (green) remained on the surface coating after 30 min of buffer rinse, as shown in FIG. 15C, due to its high affinity to fibronectin.

The result shows a shear stress about 3 dyne/cm² is sufficient to remove the non-specific cells from the releasable composition.

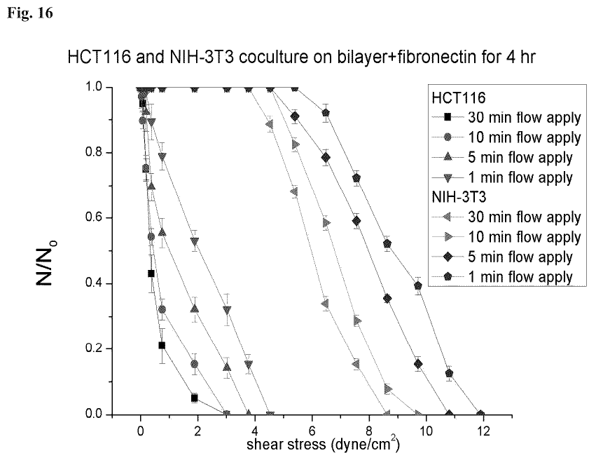

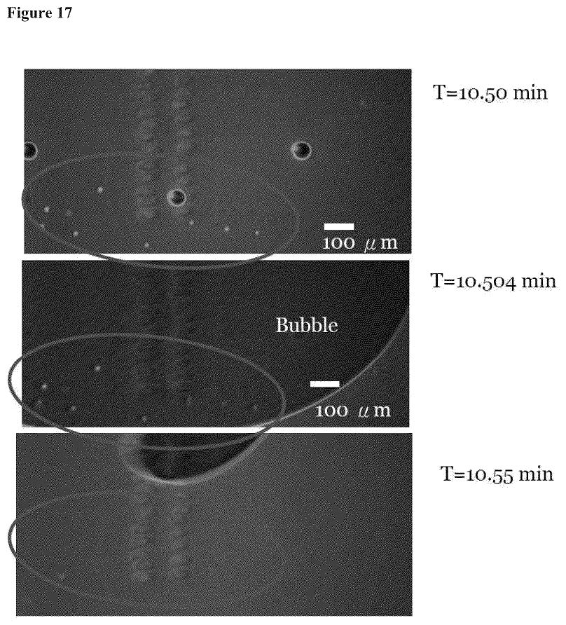

FIG. 16 summarizes the respective shear stress and flushing time for the HCT116 and NIH-3T3 cell populations (non-specific cells). To remove HCT116 cells from the releasable composition of the surface coating, the shear stress is about 3 to about 4.5 dyne/cm². To remove NIH-3T3 cells from the releasable composition of the surface coating, the shear stress is about 8.5 to about 12 dyne/cm² (N/NO is the percentage of the cells remains attached to the surface coating using various shear stresses. N is the final cell number and NO is the initial cell number.)

### Example 6: Release of CTCs from the Surface Coating

The captured HCT116 cancer cells on the surface coating in Example 3 were released by introducing air bubbles. FIG. 17 shows HCT116 cells in the red circle were removed from the surface coating within 3 seconds of introducing air bubbles.

### Example 7: Culture of Released CTCs From the Surface Coating

The captured CTCs were incubated with 5 mM of EDTA at 37 C for 5 to 10 min and released by flowing a culture medium into the sealed channel of the microfluidic chip. A total of 18 colo205 cells were released from this procedure. The released colo205 cells, together with a serum-containing culture medium and antibiotics (penicillin+streptomycin+gentamicin), were placed into a 48-well tissue cultured polystyrene plate for cultivation.

FIGS. 18A-18C show a portion of 18 colo205 cells on day 1, on day 10 and day 14 respectively. This study demonstrates the released colo205 cells retained their viability for subsequent cell culture.

### Example 8: Capture CTCs through a CTC Filtration Device

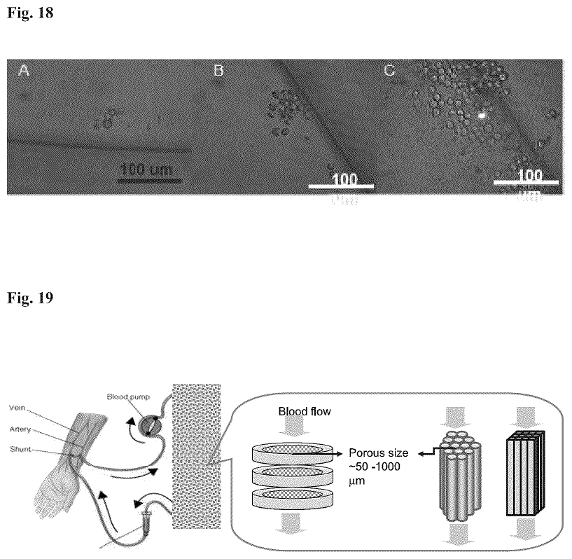

Any membranes, tubes, capillaries, beads, nanoparticles or channels can be coated with the surface coating of the present invention. FIG. 19 illustrates schematically a filtration device, wherein the filtration filter is coated with the surface coating of the present invention. The filter could accommodate high volume blood flow and capture a biological substance for a diagnostic or therapeutic purpose. To access the patient's blood or body fluid, a catheter can be inserted into the patient's vein or fistula and the patient's blood flows through the CTC filtration device, wherein the surface coating on the filters captures the CTCs. The filtered blood flows back to the patient.

### Example 9: Capture CTCs through a Biotinylated EpAb4-I Antibody

The binding specificity of biotinylated OC9801 antibody, biotinylated EpAb4-1 antibody and biotinylated EpCam antibody (commercially available from R&D system, USA) were examined using the HCT116 (colorectal) CTCs and SAS (tongue) CTCs.

The CTCs were spiked in a buffer solution (about 10⁵ CTCs/ml). The CTC-spiked buffer solution was introduced to the surface coatings with the following bioactive composition: biotinylated OC9801 antibody, biotinylated EpAb4-1 antibody, biotinylated EpCam antibody and IgG antibody.

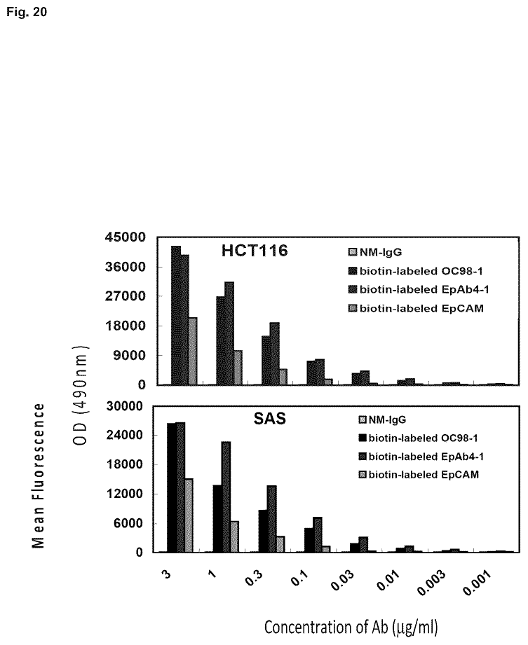

The CTC binding specificy of the antibodies was determined by colorimetric method, by measuring the absorption optical density at 490 nm. FIG. 20 shows biotinylated EpAb 4-1 is effective in capturing HCT116 CTCs and SAS CTCs.

1.-63. (canceled)

64. A microfluidic chip for selectively enriching rare cells, the microfluidic chip comprising a substrate and a surface coating for capturing rare cells, wherein the surface coating comprises:
(i) a bioactive composition which selectively binds to the rare cells, wherein the bioactive composition comprises an antibody; and
(ii) a non-fouling composition that reduces binding of non-specific cells or adsorption of serum proteins compared to a surface coating lacking the non-fouling composition, wherein the non-fouling composition comprises a lipid layer; and
wherein the antibody is non-covalently associated with the non-fouling composition.

65. The microfluidic chip of claim 64, wherein the surface coating is non-covalently associated with the substrate.

66. The microfluidic chip of claim 65, wherein the non-fouling composition is disposed between the bioactive composition and the substrate.

67. The microfluidic chip of claim 65, wherein the non-fouling composition is coupled to the substrate by a linker.

68. The microfluidic chip of claim 64, wherein the antibody is non-covalently associated with the non-fouling composition via a non-covalent interaction selected from the group consisting of hydrogen bonding, electrostatic interaction, hydrophilic-hydrophilic interaction, polar-polar interaction, magnetic force, and combinations thereof.

69. The microfluidic chip of claim 64, wherein the lipid layer is a lipid bilayer.

70. The microfluidic chip of claim 69, wherein the lipid bilayer is a supported lipid bilayer.

71. The microfluidic chip of claim 64, wherein the substrate comprises a series of microstructures configured to interact with cells, wherein the series of microstructures are perpendicular to a flow direction of the microfluidic chip.

72. The microfluidic chip of claim 71, wherein the series of microstructures comprises microstructures arranged in a linear pattern such that microstructures in a first pair of adjacent rows have a distance separating the adjacent rows, thereby forming a first gap, and wherein said first gap is not in line with a second gap formed by a second pair of adjacent rows, wherein said first gap and second gap are in adjacent columns.

73. The microfluidic chip of claim 64, wherein the antibody is a biotinylated antibody

74. The microfluidic chip of claim 64, wherein the antibody is an EpCAM antibody.

75. The microfluidic chip of claim 74, wherein the EpCAM antibody comprises a heavy chain and a light chain, wherein (a) the heavy chain comprises CDR1, CDR2, and CDR3 of SEQ ID NO: 1, and (b) the light chain comprises CDR1, CDR2, and CDR3 of SEQ ID NO: 2.

76. The microfluidic chip of claim 64, further comprising a syringe pump configured to apply buffer at a flow rate configured to release non-specific cells from the non-fouling layer without releasing cells selectively bound to the bioactive composition.

77. The microfluidic chip of claim 64, further comprising a syringe pump configured to aid rinsing the microfluidic chip with a buffer at a shear force of about 2.5 to about 10 dyne/cm².

78. The microfluidic chip of claim 64, wherein the bioactive composition or the surface coating is configured to be removed from the substrate by flow of a buffer comprising bubbles.

79. The microfluidic chip of claim 64, wherein the surface coating is configured such that flow of a buffer at shear force of less than about 50 dyne/cm² releases non-specific cells from the non-fouling layer without releasing cells selectively bound to the bioactive composition.

80. The microfluidic chip of claim 79, wherein the surface coating is configured such that flow of a buffer at a shear force of from about 2.5 dyne/cm² to about 10 dyne/cm² releases non-specific cells from the non-fouling layer without releasing cells selectively bound to the bioactive composition.

81. The microfluidic chip of claim 64, wherein the surface coating is configured such that flow of a buffer at a shear force of 50 dyne/cm² or more releases the bioactive composition or the surface coating from the substrate.

82. A method of making a microfluidic chip for use in selectively enriching rare cells, the method comprising applying a bioactive composition which selectively binds to the rare cells to a non-fouling layer associated with a substrate of the microfluidic chip, wherein the non-fouling layer comprises a lipid layer and the bioactive composition comprises an antibody, wherein the antibody non-covalently associates with the non-fouling layer.

83. A method of collecting rare cells from a biological sample, the method comprising:
(a) contacting the biological sample to a microfluidic chip comprising a substrate and a surface coating for the capturing rare cells, wherein the surface coating comprises:
(i) a bioactive composition which selectively binds to the rare cells, wherein the bioactive composition comprises an antibody; and
(ii) a non-fouling composition that reduces binding of non-specific cells or adsorption of serum proteins compared to a surface coating lacking the non-fouling composition;
(b) binding the rare cells to the antibody;
(c) flowing a buffer at shear stress of from about 2.5 dyne/cm² to about 12 dyne/cm² through the microfluidic chip, thereby releasing non-specific cells from the non-fouling layer without releasing the rare cells bound to antibody from the microfluidic chip, wherein the antibody remains non-covalently associated with the non-fouling lipid layer; and
(d) flowing a buffer at shear stress at least 50 dyne/cm² through the microfluidic chip, thereby releasing the rare cells and the bioactive composition or the surface coating from the substrate.


SEQUENCE LISTING (2 sequences)

SEQ ID NO: 1 - protein, 116 aa, synthetic construct
  REGION 1..116: note = synthetic anti-epithelial cell adhesion molecule (EpCAM) membrane protein antibody EpAb4-1 heavy chain V-H9 domain
  PEPTIDE 1..25: note = framework region 1 (FW1)
  PEPTIDE 26..35: note = complementarity-determining region 1 (CDR1)
  PEPTIDE 36..50: note = framework region 2 (FW2)
  PEPTIDE 51..58: note = complementarity-determining region 2 (CDR2)
  PEPTIDE 59..98: note = framework region 3 (FW3)
  PEPTIDE 99..105: note = complementarity-determining region 3 (CDR3)
  PEPTIDE 106..116: note = framework region 4 (FW4)
QIQLVQSGPELKKPGETVKISCKASGYTFTNYGMNWVKQAPGKGLKWMGWINTYTGEPTYGDDFKGRFAFSLETSASTAYLQINNLKNEDTATYFCARFGRSVDFWGQGTSVTVSS

SEQ ID NO: 2 - protein, 112 aa, synthetic construct
  REGION 1..112: note = synthetic anti-epithelial cell adhesion molecule (EpCAM) membrane protein antibody EpAb4-1 light chain V-kappa24/25 domain
  PEPTIDE 1..23: note = framework region 1 (FW1)
  PEPTIDE 24..39: note = complementarity-determining region 1 (CDR1)
  PEPTIDE 40..54: note = framework region 2 (FW2)
  PEPTIDE 55..61: note = complementarity-determining region 2 (CDR2)
  PEPTIDE 62..93: note = framework region 3 (FW3)
  PEPTIDE 94..102: note = complementarity-determining region 3 (CDR3)
  PEPTIDE 103..112: note = framework region 4 (FW4)
DIVMTQAAFSNPVTLGTSASISCRSSKSLLHSNGITYLYWYLQKPGQSPQLLIYHMSNLASGVPDRFSSSGSGTDFTLRISRVEAEDVGIYYCAQNLENPRTFGGGTKLEIK